(19) United States
(12) Patent Application Publication
(10) Pub. No.: US 2026/0144829 A1
(43) Pub. Date: May 28, 2026

(54) SYNBIOTIC COMPOSITION, METABOLITES, AND METHOD OF PREVENTING AND TREATING OBESITY-RELATED DISEASE

(71) Applicant: Cheng-Chih HSU, TAIPEI (TW)

(72) Inventors: Cheng-Chih HSU, TAIPEI (TW); Qiang LYU, TAIPEI (TW); Hsin-Bai ZOU, TAIPEI (TW); Rou-An CHEN, TAIPEI (TW)

(21) Appl. No.: 18/841,391

(22) PCT Filed: May 6, 2024
(86) PCT No.: PCT/CN2024/091256

§ 371 Date: Aug. 26, 2024

Related U.S. Application Data
(60) Provisional application No. 63/500,281, filed on May 5, 2023.

(51) Int. Cl. A61K 35/745; A61K 31/198; A61K 31/353; A61K 31/4015; A61K 31/7076; A61K 36/77; A61K 38/05; A61P 1/16; A61P 3/04; A61P 3/06; A61P 3/10

(52) U.S. Cl. CPC A61K 35/745; A61K 31/198; A61K 31/353; A61K 31/4015; A61K 31/7076; A61K 36/77; A61K 38/05; A61P 1/16; A61P 3/04; A61P 3/06; A61P 3/10

(57) ABSTRACT

A synbiotic composition is provided, including lychee polyphenols, Bifidobacterium longum and methionine, in which the Bifidobacterium longum includes Bifidobacterium longum BCRC 910812, BCRC 12585, BCRC 14602, BCRC11847, DSMZ20104, BCRC14607, BCRC11844, BCRC11846, BCRC14601, BCRC14606, BCRC14604, BCRC12584, or a combination thereof. Also provided is a synbiotic composition for preventing and treating obesity-related diseases. Also provided are metabolites for preventing and treating obesity-related diseases.

### CROSS-REFERENCE TO RELATED APPLICATION

This application is a National Stage Entry of International application No. PCT/CN2024/091256, filed May 6, 2024 which claims priority to U.S. Provisional Application Ser. No. 63/500,281, filed on May 5, 2023, which is herein incorporated by reference in its entirety.

### BACKGROUND

### Field of Invention

The present invention relates to a composition, metabolites, and method thereof. More particularly, the present invention relates to a synbiotic composition, metabolites, and method of preventing and treating obesity-related disease.

### Description of Related Art

The global prevalence of obesity has nearly tripled since 1975, primarily because of unhealthy dietary habits. The growing prevalence of obesity has emerged as a significant global public health concern due to its association with an elevated susceptibility to several chronic diseases.

Dietary patterns and their corresponding gut microbiota profile are associated with a variety of health conditions. For instance, a western-style diet, which is typically high in fat, and animal protein, can increase the abundance of trimethylamine (TMA)-producing bacteria in the gut, which is associated with an increased risk of cardiovascular disease and other health conditions.

Therefore, how to provide a composition for preventing and treating obesity-related disease, the related art really needs to be improved.

### SUMMARY

The present disclosure provides a synbiotic composition, comprising: lychee polyphenols; Bifidobacterium longum comprising Bifidobacterium longum BCRC910812, BCRC12585, BCRC14602, BCRC11847, DSMZ20104, BCRC14607, BCRC11844, BCRC11846, BCRC14601, BCRC14606, BCRC14604, BCRC12584, or a combination thereof; and methionine.

In some embodiments, the lychee polyphenols comprise procyanidin, epicatechin, or a combination thereof.

In some embodiments, a weight percentage of the lychee polyphenols is from 1% to 30%, a weight percentage of the Bifidobacterium longum is from 10% to 50%, and a weight percentage of the methionine is from 0.5% to 20%, based on a total weight of the synbiotic composition.

In some embodiments, the Bifidobacterium longum is a live bacteria.

In some embodiments, a content of the Bifidobacterium longum is from 1×10⁹ CFU/g to 1×10¹¹ CFU/g.

The present disclosure also provides a method of preventing and treating obesity-related disease, comprising administering to a subject in need thereof an effective amount of synbiotic composition comprising lychee polyphenols, Bifidobacterium longum, and methionine, wherein the Bifidobacterium longum comprises Bifidobacterium longum BCRC910812, BCRC12585, BCRC14602, BCRC11847, DSMZ20104, BCRC14607, BCRC11844, BCRC11846, BCRC14601, BCRC14606, BCRC14604, BCRC12584, or a combination thereof.

In some embodiments, the lychee polyphenols comprise procyanidin, epicatechin, or a combination thereof.

In some embodiments, a weight percentage of the lychee polyphenols is from 1% to 30%, a weight percentage of the Bifidobacterium longum is from 10% to 50%, and a weight percentage of the methionine is from 0.5% to 20%, based on a total weight of the synbiotic composition.

In some embodiments, the Bifidobacterium longum is a live bacteria.

In some embodiments, the obesity is caused by diet.

In some embodiments, the obesity-related disease is selected from the group consisting of type 2 diabetes, hyperglycemia, glucose intolerance, dyslipidemia, insulin resistance, hyperinsulinemia, fatty liver disease, cardiovascular disease, stroke, cancer, and a combination thereof.

In some embodiments, the composition is a food composition or a pharmaceutical composition.

In some embodiments, the pharmaceutical composition in a dosage form for oral administration or topical administration.

The present disclosure also provides a method of inhibiting lipid accumulation, comprising administering to a subject in need thereof an effective amount of metabolites, wherein the metabolites comprise 5′-methylthioadenosine, valine, pyroglutamic acid, glutamic acid, methionine, adenosine, 3-adenosine monophosphate, pyro-glutamyl-valine, or a combination thereof.

The present disclosure also provides a method of preventing and treating obesity-related disease, comprising administering to a subject in need thereof an effective amount of metabolites, wherein the metabolites comprise 5′-methylthioadenosine, pyroglutamic acid, valine, glutamic acid, methionine, adenosine, 3-adenosine monophosphate, pyro-glutamyl-valine, or a combination thereof.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIGS. 1A to 1Q are lychee polyphenols attenuated HFD-induced obesity, improved the dysbiosis of the gut microbiota, and enriched Bifidobacterium longum, which exerted anti-obesity activity. FIGS. 1A to 1K: effect of lychee polyphenols on host metabolism and gut microbiota composition. FIG. 1A is body weight changes. FIG. 1B is body weight gain during the experimental period. FIG. 1C is weight of visceral fat. FIG. 1D is plasma triglycerides. FIG. 1E is fasting glucose. FIG. 1F is area under the curve (AUC) derived from oral glucose tolerance test (OGTT). FIG. 1G is AUC derived from insulin tolerance test (ITT). FIG. 1H is gut microbiota composition as represented by principal-coordinate analysis (PCoA) of Bray-Curtis distances. FIG. 1I is observed operational taxonomic unit (OTU). FIG. 1J is abundance of Bifidobacterium in feces. FIG. 1K is abundance of B. longum in feces. FIG. 1L is effect of B. longum metabolites on lipid accumulation in HepG2 cells. FIGS. 1M to 1Q: Effect of B. longum on host metabolism. FIG. 1M is body weight changes. FIG. 1N is body weight gain during the experimental period. FIG. 10 is fasting glucose. FIG. 1P is AUC derived from OGTT. FIG. 1Q is AUC derived from ITT. Data are means and SD. Statistical analyses were performed by one-way ANOVA with Tukey's range test (*, P<0.05; **, P<0.01; ***, P<0.001; ****, P<0.0001). FIGS. 1A to 1K: N=13, 13, 10 mice per group, while number of fecal samples in each group is 3. FIGS. 1M to 1Q: N=5, 4, 5 mice per group. Abbreviations: AUC, area under the curve; B. longum: Bifidobacterium longum; HFD, high-fat diet; NCD, normal chow diet; OGTT, oral glucose tolerance test; Phe, lychee polyphenols.

FIGS. 2A to 2L are identification of bioactive metabolite from B. longum.FIG. 2A is the workflow to identify bioactive metabolite from B. longum. FIG. 2B is effect of crude extract and fractions of B. longum on lipid accumulation in HepG2 cells (n=3). FIG. 2C is effect of 5′-methylthioadenosine (MTA) on lipid accumulation in HepG2 cells (n=3). FIG. 2D is correlation analysis of obesity-related phenotype and the abundance of bioactive metabolites in mice feces (n=9). The color and number in each column represented the Spearman r. FIGS. 2E to 2F: The abundance of methionine (FIG. 2E) and MTA (FIG. 2F) in mice feces (n=4-5). FIGS. 2G to 21: B. longum can produce 13C2H3-MTA from 13C2H3-S-adenosyl-L-methionine. FIG. 2G is the experimental design. B. longum were incubated with isotope-labelled methionine(−13C2H3) in a gradient concentration, and the bacterial metabolites was extracted for detecting isotope-labelled MTA. FIGS. 2H to 2I: Tandem mass validation (FIG. 2H) and extracted ion chromatogram (FIG. 2I) of the isotope labeled S-Adenosyl-L-methionine (m/z 403.1657). FIGS. 2J to 2K: Tandem mass validation (FIG. 2J) and extracted ion chromatogram (FIG. 2K) of the isotope labeled MTA (m/z 302.1173) produced by B. longum. FIG. 2L is the abundance of MTA from the feces of mice administered with methionine (n=4). Data are means and SD. Statistical analyses were performed by one-way ANOVA with Tukey's range test (*, P<0.05; **, P<0.01; ***, P<0.001; ****, P<0.0001). Abbreviations: AUC, area under the curve; B. longum: Bifidobacterium longum; HFD, high-fat diet; NCD, normal chow diet; OGTT, oral glucose tolerance test.

FIGS. 3A to 3K are MTA ameliorated obesity and metabolic disorders. FIG. 3A is body weight changes. FIG. 3B is body weight gain during the experimental period. FIG. 3C is weight of visceral fat. FIG. 3D is weight of subcutaneous fat. FIG. 3E is fasting glucose. FIG. 3F is plasma glucose profile measured during the OGTT. FIG. 3G is AUC derived from oral glucose tolerance test (OGTT). FIG. 3H is plasma glucose profile measured during the ITT. FIG. 3I is AUC derived from insulin tolerance test ITT. FIG. 3J is weight of liver. FIG. 3K is representative histological features of H&E stained liver tissue. Data are means and SD. Statistical analyses were performed by one-way ANOVA with Tukey's range test (*, P<0.05; **, P<0.01; ***, P<0.001; ****, P<0.0001). N=5, 4, 5 mice per group. Abbreviations: AUC, area under the curve; HFD, high-fat diet; MTA, 5′-methylthioadenosine; NCD, normal chow diet; OGTT, oral glucose tolerance test.

FIGS. 4A to 4E show the effect of lychee polyphenols, Bifidobacterium longum and methionine separately or in combination in inhibiting fat accumulation according to other embodiments of the present disclosure. FIG. 4A shows the body weight changes, p<0.0001, paired t-test; FIG. 4B shows the body fat weight changes, p<0.0001, paired t-test; FIG. 4C shows the body fat percentage changes, p<0.0001, paired t-test; FIG. 4D shows the visceral fat level changes, p<0.0001, paired t-test; FIG. 4E shows the waistline changes, p=0.002, paired t-test.

FIGS. 5A to 5B show the effect of metabolites in inhibiting fat accumulation according to other embodiments of the present disclosure. FIG. 5A shows the effect of MTA in inhibiting fat accumulation, *: p<0.05, t-test; FIG. 5B shows the effect of pyroglutamic acid in inhibiting fat accumulation, *: p<0.05, **: p<0.01, t-test.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number.

As used herein, the term “synbiotic” refers to nutritional foods or medicines that combine prebiotics and probiotics, and can also add postbiotic precursors to promote human health.

As used herein, the term “prebiotic” refers to substances that can be decomposed, utilized by probiotics, and promote the growth of probiotics, thereby producing beneficial effects on health.

As used herein, the term “postbiotic” refers to an inanimate microbial secretion or fragment product that provides physiological benefits to the host.

In some embodiments of the present disclosure, the synbiotic composition s administered to an individual via oral or parenteral routes. In some embodiments of the present disclosure, the synbiotic composition is formulated for administration to an individual in an oral dosage form selected from the group consisting of solutions, suspensions, emulsions, powders, lozenges, pills, syrups, lozenges, tablets, chewable gums and capsules.

In some embodiments, a pharmaceutical acceptable carrier includes, but is not limited to water, alcohols, glycol, preserving agents, antioxidants, solvent, emulsifier, suspending agent, decomposer, binding agent, excipient, stabilizing agent, chelating agent, diluent, gelling agent, preservative, lubricant, absorption enhancers, active agents, humectants, odor absorbers, fragrances, pH adjusting agents, occlusive agents, emollients, thickeners, solubilizing agents, penetration enhancers, anti-irritants, colorants, propellants, surfactant, or other similar or applicable carriers of the present invention.

In some embodiments of the present disclosure, the synbiotic composition can be a food composition. For example, added to edible materials in the form of food additives to prepare a food product for human or animal consumption. Food composition includes, but is not limited to, general foods, health foods, beverages, nutritional supplements, dairy products or feeds, etc. In some examples of oral dosage forms, the synbiotic composition may optionally include pharmaceutical and food-acceptable carriers, excipients and/or additives. In other examples, the dosage form of the complex probiotic composition may include, but is not limited to, powders, tablets, granules, suppositories, microcapsules, ampoules, liquid sprays or suppositories.

In some embodiments of the present disclosure, the synbiotic composition includes lychee polyphenols, Bifidobacterium longum and methionine.

In some embodiments, a weight percentage of the lychee polyphenols is from 1% to 30%, a weight percentage of the Bifidobacterium longum is from 10% to 50%, and a weight percentage of the methionine is from 0.5% to 20% based on a total weight of the synbiotic composition. In which the weight percentage of the lychee polyphenols is from 1% to 30%, for example, 2%, 3%, 4%, 5%, 6%, 7%, 8%, 9%, 10%, 11%, 12%, 13%, 14%, 15%, 16%, 17%, 18%, 19%, or any value between any two of these values. In which the weight percentage of the Bifidobacterium longum is from 10% to 50%, for example, 15%, 20%, 25%, 30%, 35%, 40%, 45%, or any value between any two of these values. In which the weight percentage of the methionine is from 0.5% to 20%, for example, 0.6%, 0.7%, 0.8%, 0.9%, 1%, 2%, 4%, 6%, 8%, 10%, 12%, 14%, 16%, 18%, or any value between any two of these values.

In some embodiments, a content of the Bifidobacterium longum is from 1×10⁹ CFU/g to 1×10¹¹ CFU/g, for example, 2×10⁹ CFU/g, 3×10⁹ CFU/g, 4×10⁹ CFU/g, 5×10⁹ CFU/g, 6×10⁹ CFU/g, 7×10⁹ CFU/g, 8×10⁹ CFU/g, 9×10⁹ CFU/g, 1×10¹⁰ CFU/g, 2×10¹⁰ CFU/g, 3×10¹⁰ CFU/g, 4×10¹⁰ CFU/g, 5×10¹⁰ CFU/g, 6×10¹⁰ CFU/g, 7×10¹⁰ CFU/g, 8×10¹⁰ CFU/g, 9×10¹⁰ CFU/g, or any value between any two of these values.

In some embodiments of the present disclosure provides a use of metabolites in the manufacture of a composition to inhibit lipid accumulation, in which the metabolites comprise 5′-methylthioadenosine, valine, pyroglutamic acid, glutamic acid, methionine, adenosine, 3-adenosine monophosphate, pyro-glutamyl-valine, or a combination thereof.

In some embodiments of the present disclosure provides a use of metabolites in the manufacture of a composition to prevent and treat obesity-related disease, wherein the metabolites comprise 5′-methylthioadenosine, valine, pyroglutamic acid, glutamic acid, methionine, adenosine, 3-adenosine monophosphate, pyro-glutamyl-valine, or a combination thereof.

A number of examples are provided herein to elaborate the synbiotics composition of the instant disclosure. However, the examples are for demonstration purpose alone, and the instant disclosure is not limited thereto.

### EXAMPLE

### Example 1 Lychee Polyphenols Attenuated HFD-Induced Obesity and Improved the Dysbiosis of the Gut Microbiota

Lychee fruit (Litchi chinensis Sonn.) was obtained from the local market in Taipei, Taiwan. After removing the peel and seed manually, the pulp was frozen in liquid nitrogen and dehydrated by a freeze dryer. Finally, the pulp was grounded into fine powder. The dried powder (300 g) of Lychee pulp was extracted with 70% methanol aqueous solution (2.4 L) using a ultrasonicator (30 W, 60 kHz) for 30 min to obtain extracts. After filtration of the extracts through filter papers, the residual compounds were further extracted twice using the same protocol to obtain filtrates. Methanol in all filtrates was removed using a rotary evaporator (at 45° C.). Polysaccharides in the water filtrates were removed by an Oasis HLB column (20 cc/1 g, Waters, Milford, MA, USA). The remaining compounds were spun down to obtain crude extracts. The phenolic compounds of lychee in the crude extracts were analyzed by the Global Natural Products Social (GNPS), which is a molecular networking method that relies on the concept that molecules with comparable chemical structures exhibit similar MS/MS fragmentation patterns, and the crude extracts includes lychee polyphenols, in which lychee polyphenols includes procyanidin (procyanidin B2), (-)-epicatechin (EC), quercetin rhamnosyl-rutinoside, kaempferol rhamnosyl-rutinoside, isorhamnetin rhamnosyl-rutinoside, rutin, kaempferol rutinoside, and isorhamnetin rutinoside.

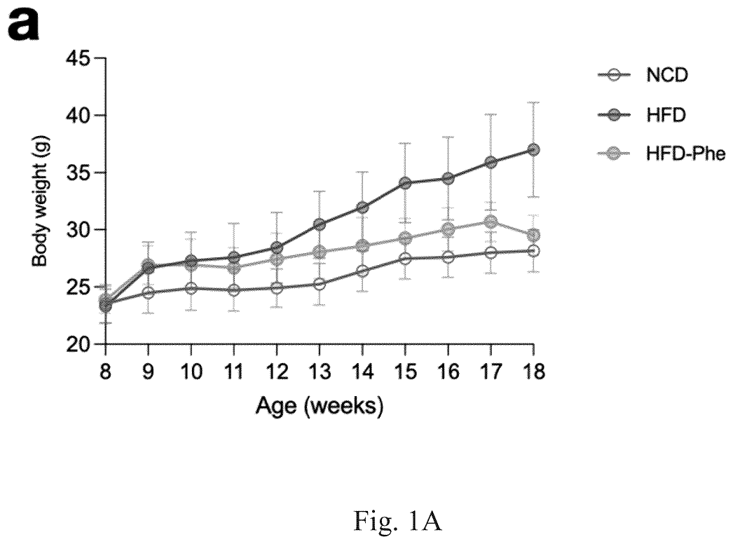

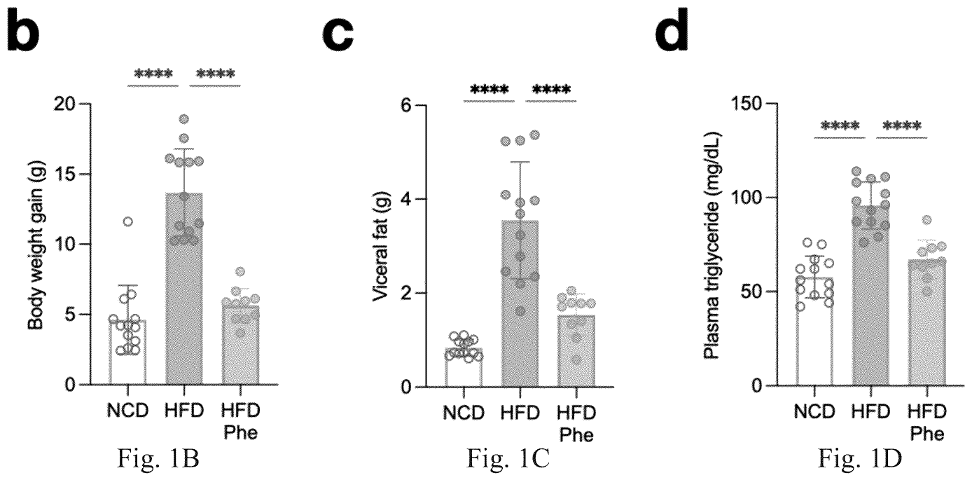

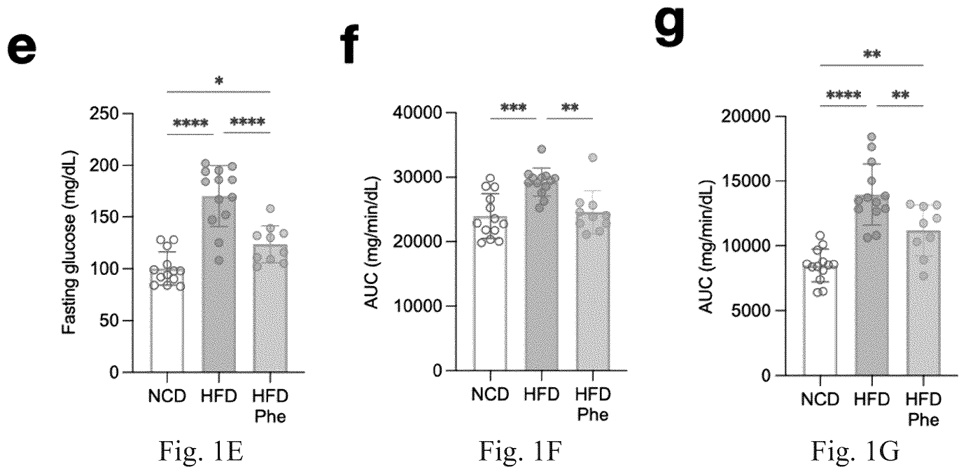

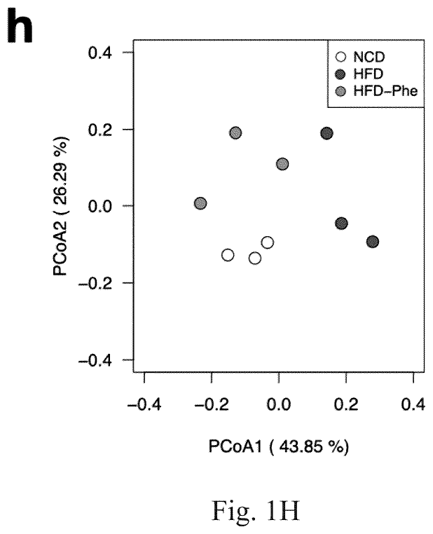

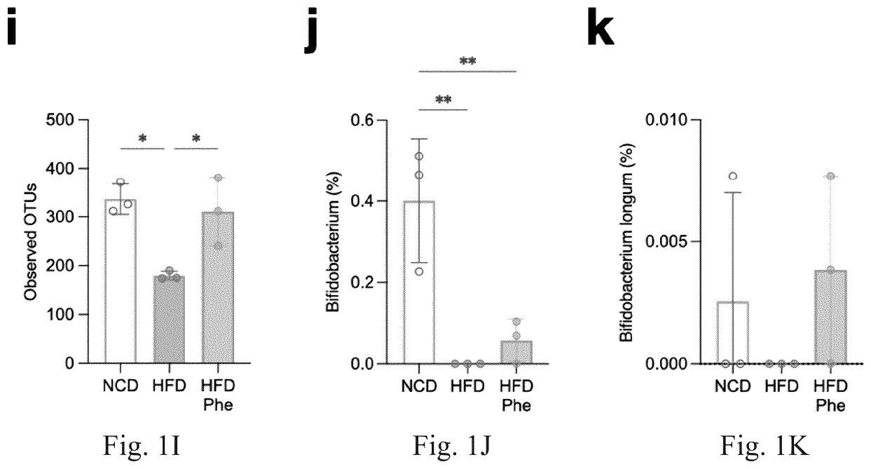

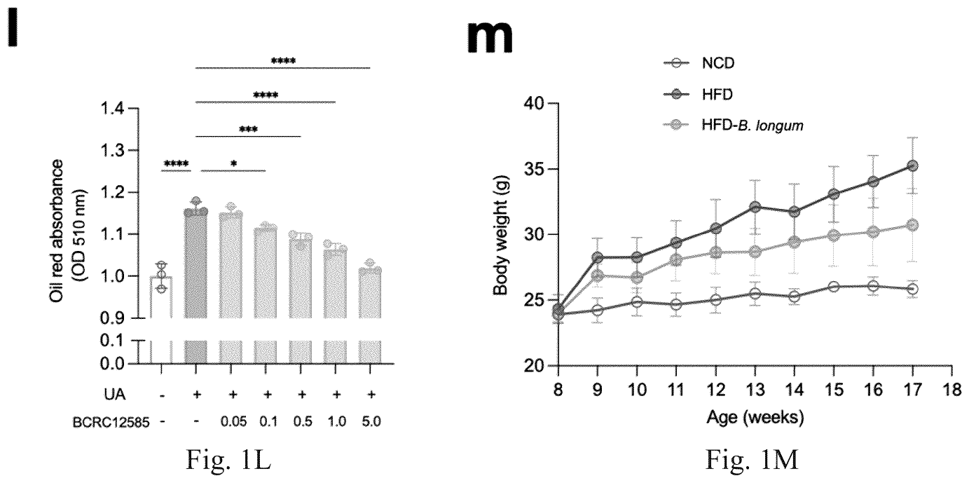

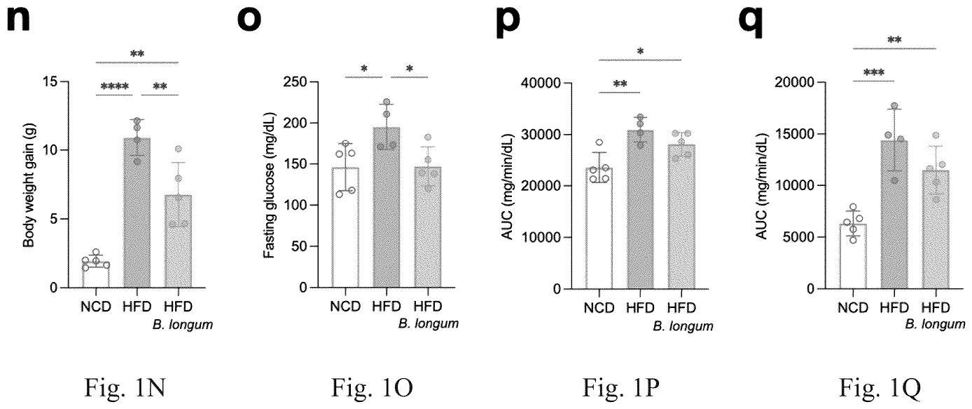

We found that lychee polyphenols alleviated high-fat diet-induced obesity, decreased hyperlipidaemia, ameliorated glucose tolerance and insulin sensitivity (FIGS. 1A to 1G). Administration of lychee polyphenols enriched the diversity and rectified the dysbiosis of microbiota caused by HFD (FIGS. 1H and 1I). Moreover, lychee polyphenols enriched the abundance of Bifidobacterium spp. and marginally increased Bifidobacterium longum (FIGS. 1J and 1K).

### Example 2 Bifidobacterium longum Exerted Anti-Obesity Activity

Next, we thought to explore the regulatory capacity of Bifidobacterium metabolites on host metabolism. To this end, we purchased all the commercial Bifidobacterium strains (11 strains: BCRC12585, BCRC14602, BCRC11847, DSMZ20104, BCRC14607, BCRC11844, BCRC11846, BCRC14601, BCRC14606, BCRC14604, BCRC12584; BCRC refers to Bioresource Collection and Research Center of Taiwan Food Industry Research and Development Institute, and DSMZ refers to German Collection of Microorganisms and Cell Cultures GmbH) that were available in Taiwan and colonized then to C57BL/6 germ-free mice. The gnotobiotic mice were orally inoculated with 1×10⁹ CFUs (colony-forming units) of Bifidobaterium (11 strains evenly mix) in 0.2 mL of sterile PBS once a week, and the germ-free mice was gavaged with 0.2 mL of sterile PBS as the control group. All the mice were fed with a high-fat diet (OpenSource Diets™, D12492, 60% calories from fat). To investigate whether the metabolites of Bifidobacterium could attenuate obesity, the mouse feces (100 mg) were extracted with a 70% methanol aqueous solution (1 mL) containing cholic acid-d4 (2 ppm) as an internal standard. After 30 minutes of homogenization using an ultrasonicator (30 W, 60 kHz), the extracts were centrifuged at 12,000 rpm for 10 min at 4° C., and the supernatants as metabolites were collected and dried.

The lipid accumulation assay was done. Briefly, HepG2 cells at 70% confluence were incubated in RPMI 1640 medium containing 120 μg/mL uric acid for 24 hours, which was used to increase the lipid accumulation. After being washed by PBS buffer, the cells were maintained in new RPMI 1640 medium containing the metabolites (5.0, 1.0, 0.5, 0.1, and 0.05 μg/mL, the dried metabolites were redissolved in RPMI 164 medium) and 120 μg/mL uric acid for another 24 hours. Cells maintained in pure RPMI 1640 medium were employed as control. The result suggests that by testing the lipid accumulation activity with HepG2 cells, metabolites extracted from Bifidobacterium-fed mice's feces significantly decreased lipid accumulation in a dose-dependent manner (figure not shown). Meanwhile, metabolites from each strain of Bifidobacterium exerted anti-obesogenic activity as well (FIG. 1L is B. longum BCRC12585, others not shown). These findings suggest that the anti-obesity effect of polyphenols could be attributed to the enrichment of Bifidobacterium. Since we attempted to identify specific chemicals produced by probiotic microorganisms that have therapeutic potential and could be easily scale up in industry, Bifidobacterium. longum. subsp. longum BCRC12585 (hereinafter referred to as B. longum), a commercial strain of B. longum isolated from human and easily to scale up, was selected to further explore the bioactive metabolites in a mice model.

Next, 10⁸ CFU of B. longum was administered to C57BL/6 mice once a day until one week prior to the sacrifice. As a result, the mice exhibited decreased body weight and weight gain (FIGS. 1M and 1N), while visceral and subcutaneous fat were marginally decreased in B. longum-fed mice (figure not shown). Besides, mice treated with B. longum showed remarkably decreased fasting glucose (FIGS. 1O, 1P, and 1Q) and slightly improvement of both glucose and insulin tolerance (figure not shown).

### Example 3 Identification of Bioactive Metabolite Produced by B. longum

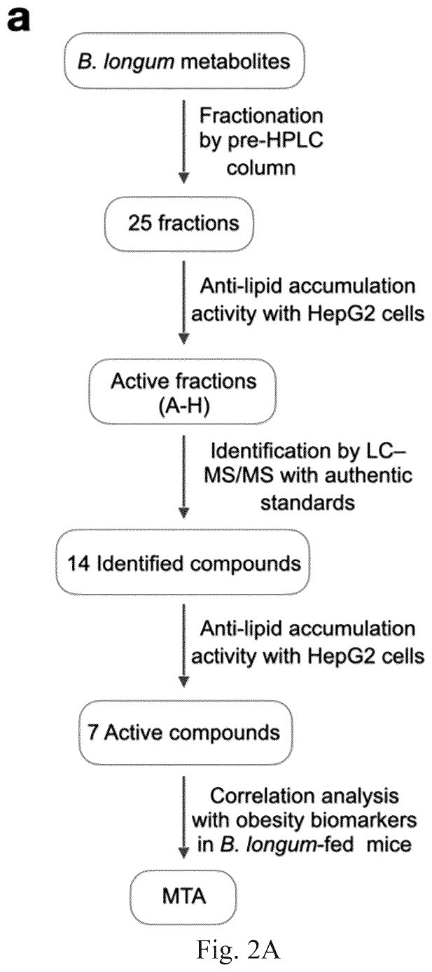

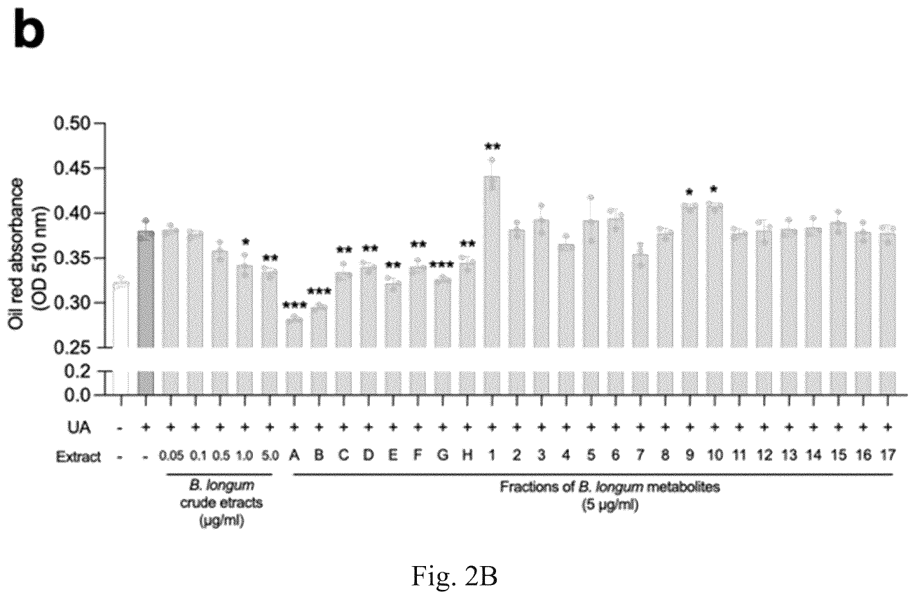

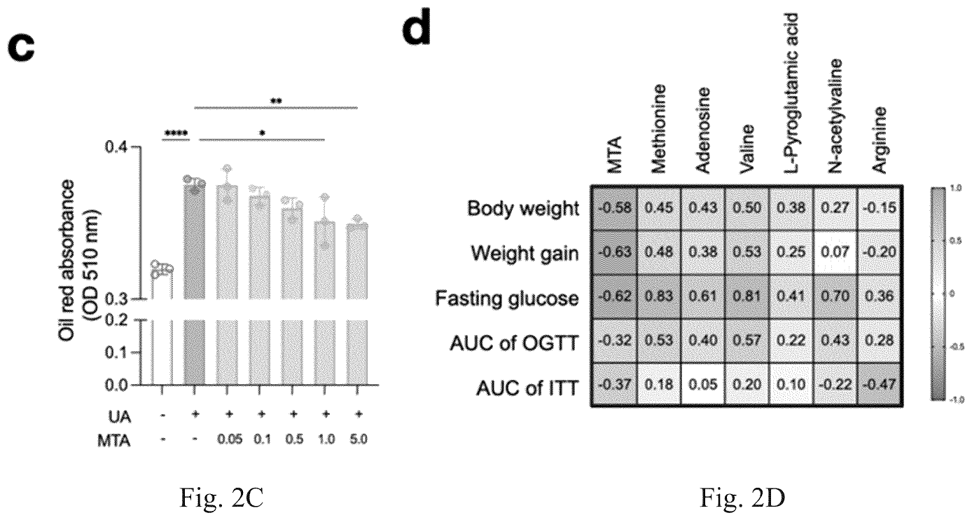

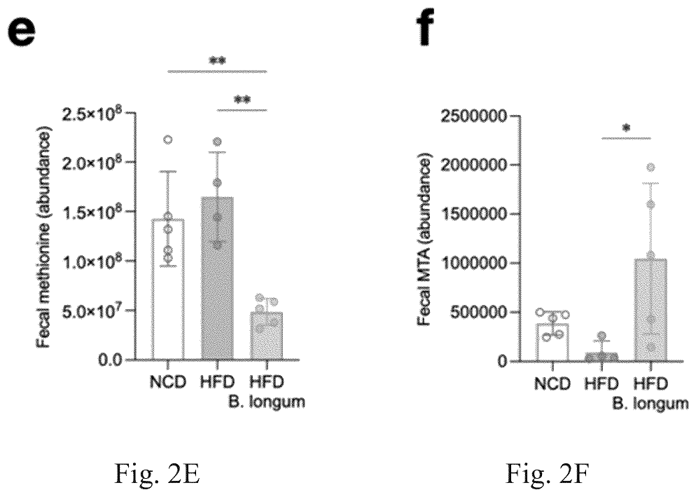

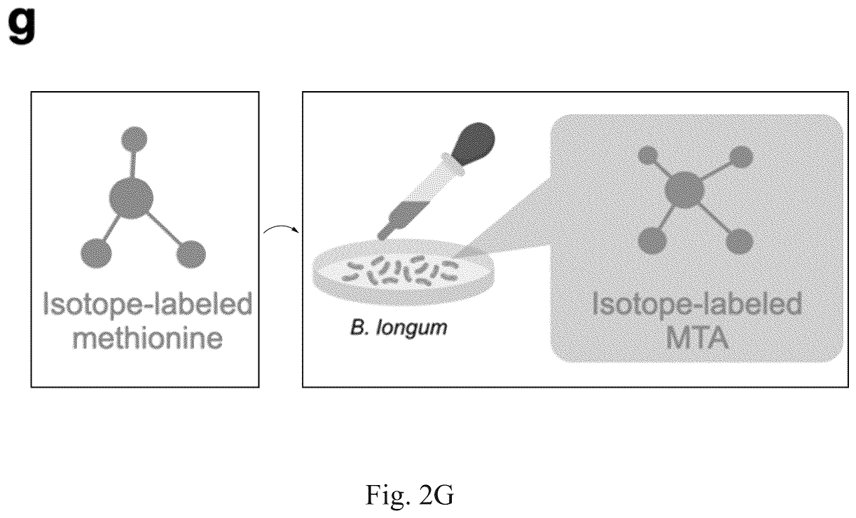

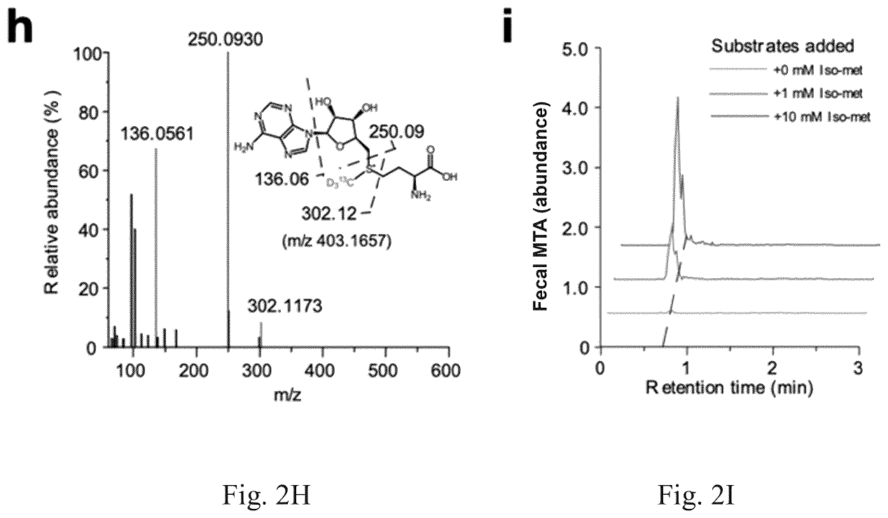

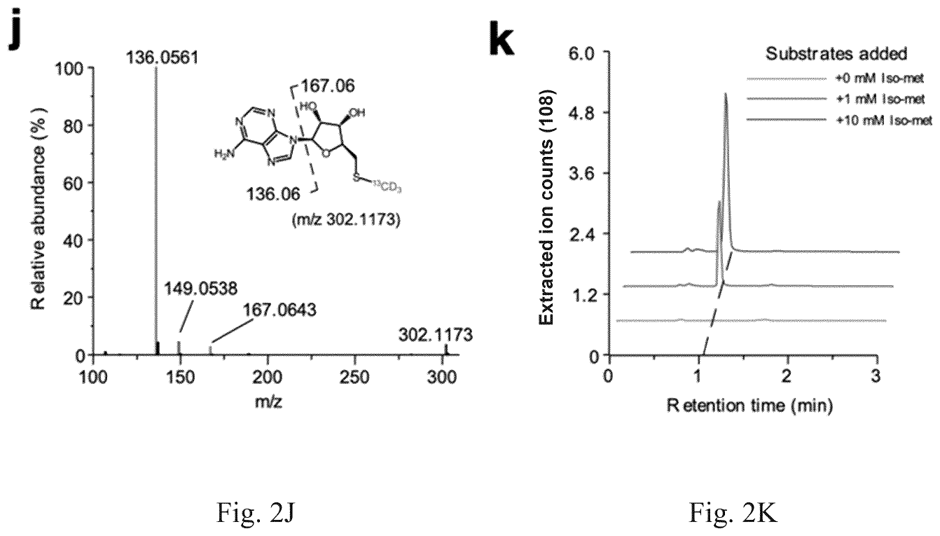

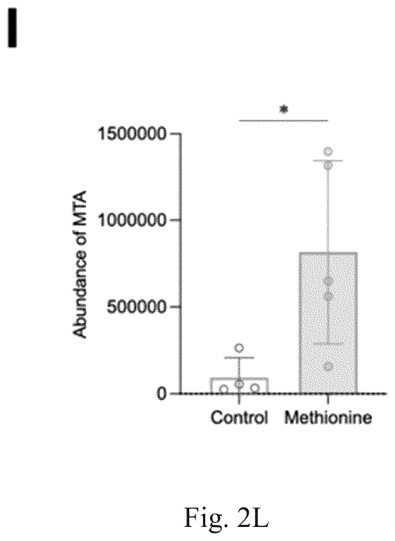

To further identify bioactive metabolites produced by B. longum, we applied a fractionation approach followed by liquid chromatography-mass spectrometry (LC-MS) coupled with in vitro screening strategy (FIG. 2A). Briefly, the extraction method of B. longum BCRC12585 metabolites (that is, B. longum metabolites, or B. longum crude extract) is the same as in Example 2, and then twenty-five fractions derived from B. longum metabolites were prepared by pre-HPLC column, followed by individually testing the anti-lipid accumulation activity with HepG2 cells. Interestingly, fraction A to fraction H, which had a short retention time, strongly inhibited lipid accumulation (FIG. 2B), indicating that the active compounds were mostly small molecules with high polarity. To identify candidate metabolites from each active fraction, further analysis was performed by using liquid chromatography coupled with high resolution mass spectrometry (LC-MS/MS). Fourteen compounds, including nine amino acids (proline, valine, glutamine, glutamic acid (GA), arginine, pyroglutamic acid (PyroGA), methionine, isoleucine, and tryptophan), two peptides (cyclo-(His-Pro), pyro-glutamyl-valine (Pyro-glu-val)), and adenosine and its two derivatives (3-adenosine monophosphate (3-AMPP), and 5′-s-methyl-5′-thioadenosine (MTA)), were identified and further verified using authentic standards (figure not shown). Among these identified metabolites, eight of these metabolites (MTA, valine, PyroGA, GA, methionine, adenosine, 3-AMPP, Pyro-glu-val) significantly inhibited lipid accumulation in HepG2 cells (MTA as shown in FIG. 2C, and others not shown). Notably, the abundance MTA exhibited a negative correlation with most of the obesity biomarkers of mice (FIG. 2D). Collectively, we integrated in vitro, in vivo, and metabolomics data to identify the bioactive metabolites which could be produced by B. longum.

Interestingly, both MTA and its precursor methionine were identified in the active fractions of B. longum and exhibited a lipid-lowering effect in HepG2 cells (MTA as shown in FIG. 2C, and others not shown), we next investigated whether B. longum could produce MTA from methionine. The experimental method is that the animals were administrated high HFD for 17 weeks, and were started to gavage MTA 100 mg per kilogram of body weight daily (100 mg/kg/day) from the 8th week. As the precursor for MTA synthesis, methionine was decreased in the feces of mice treated B. longum, while MTA was increased (FIGS. 2E and 2F). Next, in vitro isotope-labelled experiment coupled with mass spectrometry was used to understand whether B. longum could convert methionine to MTA. Briefly, B. longum was incubated in the plate coated with 1 mL of isotope labeled methionine (Iso-met, −13C2H3) with 0, 1, and 10 mM, respectively (FIG. 2G). As a result, 13C2H3-MTA with m/z 302.1173 (M+4 ) and its intermediate products 13C2H3-S-Adenosyl-L-methionine with m/z 403.1657 (M+4 ) were detected with a dose-dependent effect (FIGS. 2H to 2K), suggesting B. longum could synthesize MTA by using methionine. Furthermore, administration of methionine increased the level of MTA in mice feces (FIG. 2L). In addition, we found that the other Bifidobacterium species used in this study, including B. breve, B. adolescentis, B. infantis, B. longum, B. bifidum, B. animalis, could also synthesize MTA with methionine (figure not shown). Here we discovered a bioactive metabolite MTA, which having anti-obesogenic potential, can be produced by Bifidobacterium via converting dietary amino acid.

### Example 4 MTA, a Methionine-Derived Metabolite Produced by B. longum, Ameliorated Obesity and Metabolic Disorders

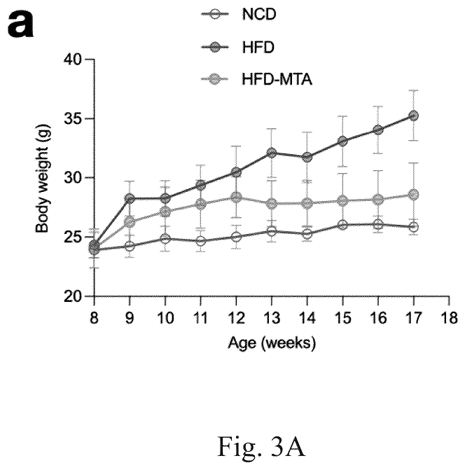

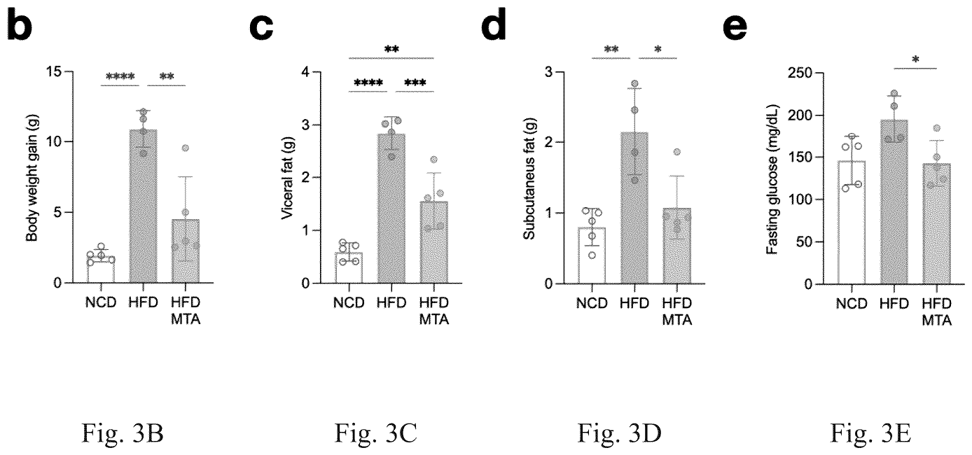

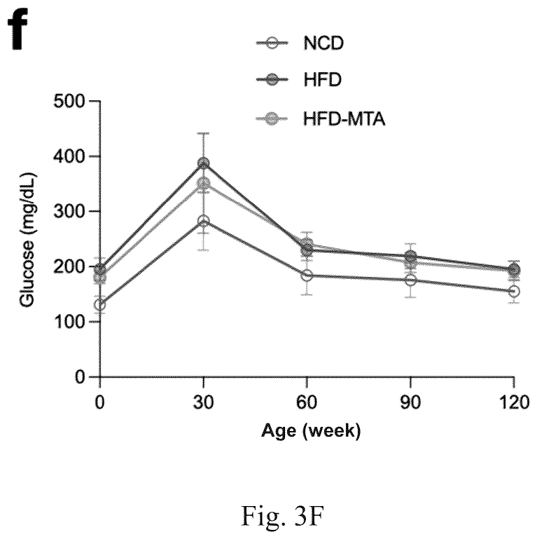

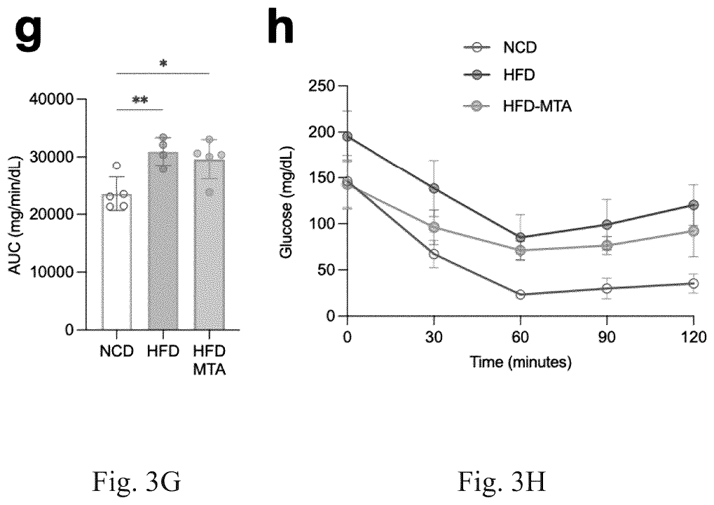

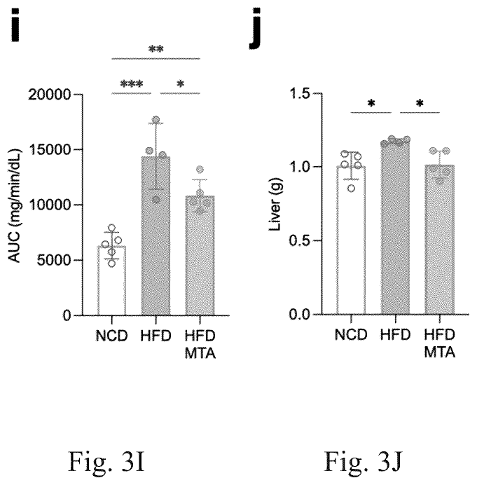

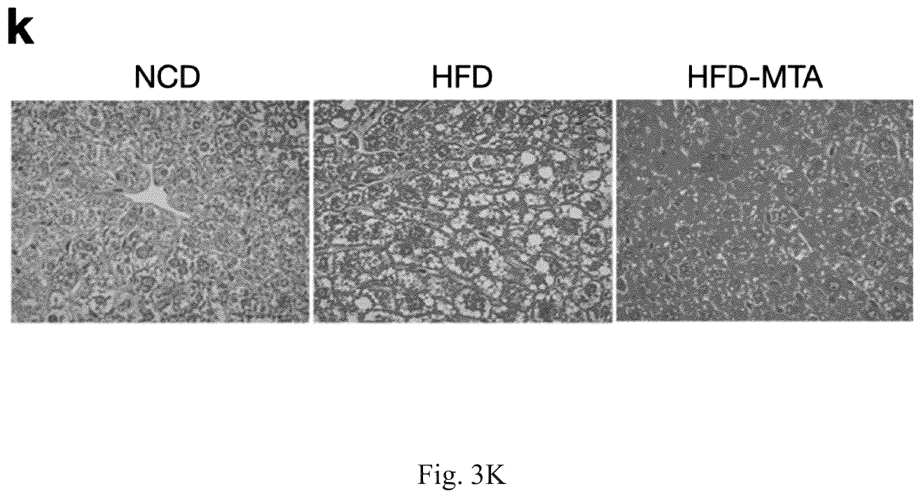

As a biosynthetic precursor for several key chemicals, MTA plays a role in energy metabolism, including the regulation of immune system, gene expression and the synthesis of important biomolecules such as DNA and proteins. Thus, we next investigated the effect of MTA on host metabolism by treating HFD-fed C57BL/6 mice with or without MTA. The experimental method is that the animals were administrated high HFD for 17 weeks, and were started to gavage MTA 100 mg per kilogram of body weight daily (100 mg/kg/day) from the 8th week. After administration of MTA, the metabolic indicators, including body weight, weight increase over the study period, visceral and subcutaneous fat mass were significantly decreased in mice (FIGS. 3A to 3E). Similar reductions were observed in glucose tolerance and insulin sensitivity (FIGS. 3F to 3I). Moreover, liver weight and liver H&E staining revealed that MTA could alleviate hepatic steatosis in obese mice (FIGS. 3J and 3K). These results indicate that MTA may effectively regulate lipid metabolism and insulin sensitivity, thereby reducing obesity and hepatic steatosis.

### Example 5 MTA Exerts Numerous Regulatory Effects on Hepatic Energy Metabolism

To obtain a more comprehensive understanding of MTA's potential as an antiobesity molecule, hepatic RNA-sequencing was performed on mice treated with both MTA and B. longum to determine its underlying mechanism for preventing obesity. The gene ontology (GO) enrichment analysis of top 30 differentially expressed molecular function revealed that MTA considerably regulated the genes involved in fatty acid metabolism (Ltb4r1, Insig2, Elov/3 and Trib3), neurotransmitter signaling (Hcn3 and Adrb2), growth hormone signaling (Enho and Arntl), leptin signaling and bile acid metabolism (figure not shown), while B. longum mainly modulated the genes involved in fatty acid metabolism, bile acid metabolism and immune response (figure not shown). Then, we conducted an in-depth analysis of the regulated genes involved in these biological functions. Notably, the expression of insulin induced gene 2(Insig2 ), which influences cholesterol metabolism, lipogenesis, and glucose homeostasis in the liver, decreased in MTA-treated mice. MTA treatment also decreased ELOVL fatty acid elongase 3 (Elov/3), which is responsible for very-long-chain fatty acid elongation. These findings suggest that suppression of Insig2 and Elovi3 by MTA may contribute to fatty acid remodeling in the liver and aid in body weight regulation. Additionally, beta-2-adrenergic receptor gene (Adrb2) and leptin receptor gene (Lepr), which promote metabolic signaling associated to energy expenditure via epinephrine and leptin, respectively, were up-regulated in MTA-treated mice. Besides, elevated expression of adropin (encoded by energy homeostasis-associated gene, Enho) in MTA-treated mice may improve glucose metabolism by enhancing glucose utilization. Likewise, overexpression of inhibin beta-A, which is encoded by the Inhba gene, can stimulate mitochondrial energy metabolism and increase energy expenditure. These results suggest that MTA could improve energy metabolism by modulating the signaling of these metabolic hormones and proteins. Collectively, these results reveal that the effect of MTA on the prevention of obesity and insulin resistance may be due to a variety of regulation activities of energy metabolism, including fatty acid biosynthesis, glucose utilization, and energy metabolism, which may be regulated by the signaling of hormones and proteins with metabolic regulatory effects.

Bile acids are regarded as important signaling molecules to regulate fatty acid, cholesterol, energy and glucose homeostasis; therefore, enhancing bile acid synthesis may be a strategy to avoid diet-induced obesity. Furthermore, the increased expression of Cyp7a1, which encodes the rate-limiting enzyme in the classic bile acid synthesis pathway, prevents high-fat diet-induced obesity, insulin resistance and atherosclerosis. Given that the expression of Cyp7a1 was up-regulated in mice treated with MTA (figure not shown), hepatic bile acid analysis was carried out to determine whether the regulated Cyp7a1 level contributes to bile acid composition. Result indicates that mice administered MTA exhibited an enhanced trend of hepatic primary bile acids. Taken together, these results suggest that MTA could increase the Cyp7a1expression level and regulate its downstream bile acid synthesis as well as fatty acid metabolism.

### Example 6 The Composition of Lychee Polyphenols, B. longum, and MTA Has Effects in Inhibiting Fat Accumulation

The synbiotic composition includes lychee polyphenols, Bifidobacterium longum and methionine. The lychee polyphenols in the synbiotic composition were purchased from commercially available Oligonol® (the main active ingredients include proanthocyanidins (proanthocyanidins A1, A2, B1, B2), catechin, epicatechin (EC), epicatechingallate (ECG), epigallocatechingallate (EGCG), epicatechin-(4β→8, 2β→O→7) eicatechin-(4β→8)-eicatechin (A2-EC), and green tea polyphenols), and content of the lychee polyphenols is 10 wt %; Bifidobacterium longum was obtained from the B. longum subsp. infantis BLI-02 strain (deposited at the Bioresource Collection and Research Center of the Taiwan Food Industry Research and Development Institute BCRC910812 (this strain is freely available for distribution), or China General Microbiological Culture Collection Center CGMCC No. 15212), and the content of the Bifidobacterium longum is 30 wt %, in which the effective live bacteria include 1×10¹⁰ CFU/g (for example, the total weight of the synbiotic composition is 0.5 g); and the content of the methionine is 2 wt % based on total weight (100% by weight) of the synbiotic composition. The synbiotic composition is prepared in capsule form, and the above weight percentage does not include the weight of the capsule.

27 voluntary subjects met the following screening conditions, 8 were male and 19 were female, and the subjects were aged between 18 and 65 years old. The implementation method of the example was to provide the subject to take the synbiotic composition for five consecutive weeks. The taking method was two capsules twice a day. Based on the caloric intake recommended in the subject's body composition analysis results, the nutritionist provided the subject with caloric and dietary control that was consistent with his or her body weight to control variables. The subjects' weight, body fat weight, body fat percentage and visceral fat level were measured using a body fat analysis (InBody® model: InBody 570, an instrument that uses bioelectric impedance (BIA) to analyze body composition) before and after taking the synbiotic composition for 5 weeks.

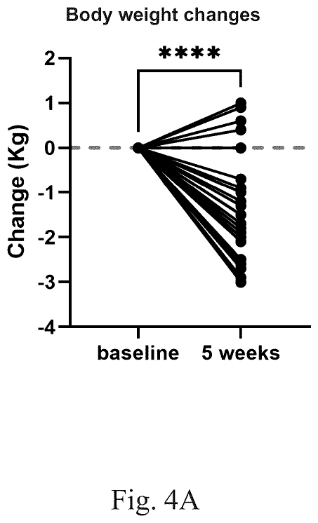

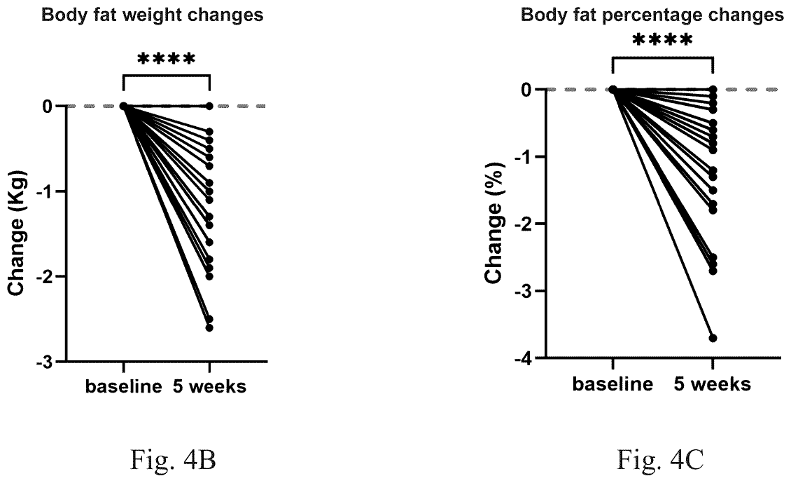

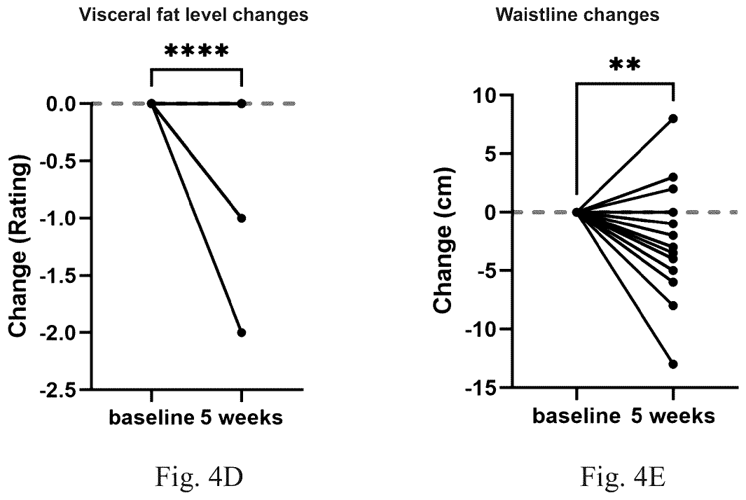

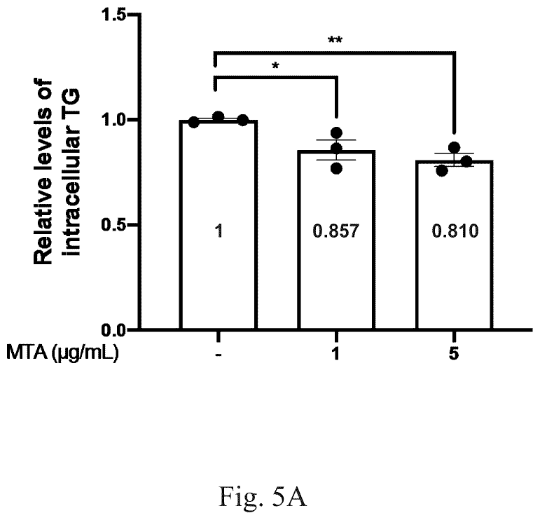

The results are shown in FIGS. 4A to 4E. FIG. 4A shows that compared with the weight before taking the synbiotic composition, the weight of the subjects decreased significantly after taking the synbiotic composition for five weeks. The average weight of the 27 subjects decreased by 1.39 kilograms. FIG. 4B shows that compared with the body fat weight before taking the synbiotic composition, the body fat weight of the subjects after taking the synbiotic composition for five weeks decreased significantly. The average body fat weight of 27 subjects decreased by 1.27 kilograms. FIG. 4C shows that compared with the body fat percentage before taking the synbiotic composition, the body fat percentage of the subjects was significantly decreased after taking the synbiotic composition for five weeks. The average body fat percentage of the 27 subjects was reduced by 1.21%. FIG. 4D shows that compared with the visceral fat level before taking the synbiotic composition, the body fat rate of the subjects was significantly reduced after taking the synbiotic composition. After five weeks of taking the synbiotic composition, the visceral fat level decreased significantly, and the average visceral fat level of 27 subjects decreased by 0.7 levels. FIG. 4E shows that compared with the waistline before taking the synbiotic composition, the waistline of the subjects after taking the synbiotic composition for five weeks decreased significantly. The average waistline decreased by 2.57 cm.

In some examples, in the synbiotic composition, the lychee polyphenols of obtained from Example 1 and the Bifidobacterium longum obtained from BCRC12585, BCRC14602, BCRC11847, DSMZ20104, BCRC14607, BCRC11844, BCRC11846, BCRC14601, BCRC14606, BCRC14604, BCRC12584, or a combination thereof can also achieve similar effects of weight loss, body fat weight reduction, body fat percentage reduction, visceral fat level reduction, and waistline reduction.

### Example 7 MTA, Pyroglutamic Acid Has Effects in Inhibiting Fat Accumulation

3T3-L1 cells were seeded on a 12-well plate at a cell density of 3×10³ cells/cm² and cultured in DMEM medium containing 10% bovine calf serum (CS), 100 U/mL penicillin and 100 μg/mL streptomycin for 3 to 4 days, the medium was replaced every 2 days until the cell density reached approximately 70% or more, and then differentiation was performed. DMEM medium containing 0.5 mM 3-isobutyl-1-methylxanthine (IBMX), 1 μM dexamethasone, 10 μg/mL insulin, 10% fetal bovine serum (FBS), 100 U/mL penicillin and 100 μg/mL streptomycin was replaced for 2 days. Then, insulin was used for induction, and DMEM containing 10 μg/mL insulin, 10% fetal bovine serum, 100 U/mL penicillin and 100 μg/mL streptomycin was replaced to induce fat accumulation in 3T3-L1 cells. At the same time during induction, different compounds (MTA, pyroglutamic acid) were treated for 2 days.

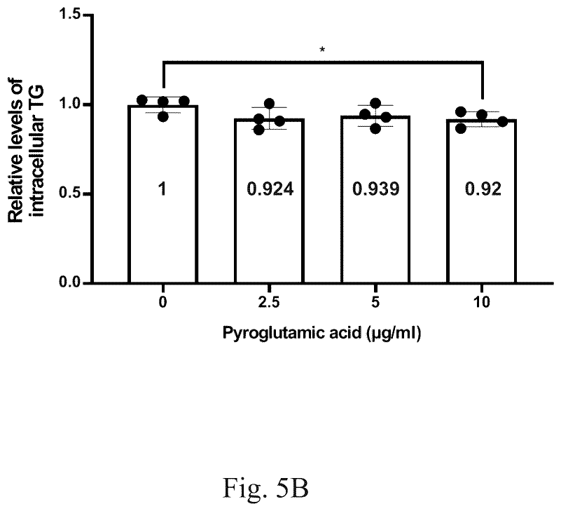

The results are shown in FIG. 5A. Regardless of the administration of 1 μg/mL or 5 μg/mL MTA, triglyceride (TG) accumulation in 3T3-L1 adipocytes was significantly inhibited. As shown in FIG. 5B, administered of 10 μg/mL MTA significantly inhibited triglyceride accumulation in 3T3-L1 adipocytes; administration of 2.5 μg/mL or 5 μg/mL MTA also had a tendency to inhibit.

In conclusion, a variety of methods disclose a molecular mechanism that polyphenols prevent obesity by enriching Bifidobacterium and MTA, a bioactive metabolite of Bifidobacterium possessing anti-obesity activity. Furthermore, polyphenols, Bifidobacterium and methionine, the precursor of MTA, can be combined as a symbiotic product as functional food or medication, with each component having anti-diabetic properties.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A synbiotic composition, comprising:
lychee polyphenols;
Bifidobacterium longum comprising Bifidobacterium longum BCRC910812, BCRC12585, BCRC14602, BCRC11847, DSMZ20104, BCRC14607, BCRC11844, BCRC11846, BCRC14601, BCRC14606, BCRC14604, BCRC12584, or a combination thereof; and
methionine.

2. The synbiotic composition of claim 1, wherein the lychee polyphenols comprise procyanidin, epicatechin, or a combination thereof.

3. The synbiotic composition of claim 1, wherein
a weight percentage of the lychee polyphenols is from 1% to 30%,
a weight percentage of the Bifidobacterium longum is from 10% to 50%, and
a weight percentage of the methionine is from 0.5% to 20%,
based on a total weight of the synbiotic composition.

4. The synbiotic composition of claim 1, wherein the Bifidobacterium longum is a live bacteria.

5. The synbiotic composition of claim 1, wherein a content of the Bifidobacterium longum is from 1×10⁹ CFU/g to 1×10¹¹ CFU/g.

6. A method of preventing and treating obesity-related disease, comprising administering to a subject in need thereof an effective amount of synbiotic composition comprising lychee polyphenols, Bifidobacterium longum, and methionine, wherein the Bifidobacterium longum comprises Bifidobacterium longum BCRC910812, BCRC12585, BCRC14602, BCRC11847, DSMZ20104, BCRC14607, BCRC11844, BCRC11846, BCRC14601, BCRC14606, BCRC14604, BCRC12584, or a combination thereof.

7. The method of claim 6, wherein the lychee polyphenols comprise procyanidin, epicatechin, or a combination thereof.

8. The method of claim 6, wherein
a weight percentage of the lychee polyphenols is from 1% to 30%,
a weight percentage of the Bifidobacterium longum is from 10% to 50%, and
a weight percentage of the methionine is from 0.5% to 20%,
based on a total weight of the synbiotic composition.

9. The method of claim 6, wherein the Bifidobacterium longum is a live bacteria.

10. The method of claim 6, wherein the obesity is caused by diet.

11. The method of claim 6, wherein the obesity-related disease is selected from the group consisting of type 2 diabetes, hyperglycemia, glucose intolerance, dyslipidemia, insulin resistance, hyperinsulinemia, fatty liver disease, cardiovascular disease, stroke, cancer, and a combination thereof.

12. The method of claim 6, wherein the composition is a food composition or a pharmaceutical composition.

13. The method of claim 12, wherein the pharmaceutical composition in a dosage form for oral administration or topical administration.

14. A method of preventing and treating obesity-related disease, comprising administering to a subject in need thereof an effective amount of metabolitesuse, wherein the metabolites comprise 5′-methylthioadenosine, pyroglutamic acid, valine, glutamic acid, methionine, adenosine, 3-adenosine monophosphate, pyro-glutamyl-valine, or a combination thereof.

15. The method of claim 14, wherein the obesity is caused by diet.

16. The method of claim 14, wherein the obesity-related disease is selected from the group consisting of type 2 diabetes, hyperglycemia, glucose intolerance, dyslipidemia, insulin resistance, hyperinsulinemia, fatty liver disease, cardiovascular disease, stroke, cancer, and a combination thereof.

17. The method of claim 14, wherein the composition is a food composition or a pharmaceutical composition.

18. The method of claim 17, wherein the pharmaceutical composition in a dosage form for oral administration or topical administration.
